(19) United States
(12) Patent Application Publication
(10) Pub. No.: US 2026/0364005 A1
(43) Pub. Date: Nov. 19, 2026

(54) UNIVERSAL FORMULATION OF ATOMIZED LIPID NANOPARTICLE FOR INHALATION

(71) Applicant: RONGCAN (SHANGHAI) BIOTECH CO., LTD, Shanghai (CN)

(72) Inventors: Xueqing ZHANG, Shanghai (CN); Yilong TENG, Shanghai (CN); Qijing CHEN, Shanghai (CN)

(21) Appl. No.: 19/127,309

(22) PCT Filed: Nov. 3, 2023
(86) PCT No.: PCT/CN2023/129753

§ 371 Date: Jul. 17, 2026

(30) Foreign Application Priority Data
Nov. 3, 2022 (CN) 202211374342.0

(51) Int. Cl. A61K 9/00; A61K 9/51; A61K 47/10; A61K 47/14

(52) U.S. Cl. CPC A61K 9/0078; A61K 9/5123; A61K 47/10; A61K 47/14

(57) ABSTRACT

The present invention relates to the field of biological pharmaceuticals, and particularly, to a universal formulation of an atomized lipid nanoparticle for inhalation. The formulation comprises: a lipid nanoparticle (LNP) dispersion system and a surface tension down-regulator added into the LNP dispersion system. The particle size range of the LNP is 70-160 nm. The mass-to-volume percentage of the surface tension down-regulator in the LNP dispersion system is 0.01-30%. It has been unexpectedly discovered that the addition of the surface tension down-regulator to the LNP dispersion system can increase the tolerance of LNPs to atomization shear, thus solving the problems of poor particle size distribution, dispersity (PDI), entrapment efficiency (EE), and stability of LNPs before and after atomization due to the atomization shear force, and achieving efficient delivery while alleviating the pain of a patient receiving an atomized inhalant.

### TECHNICAL FIELD

The present invention relates to the field of nucleic acid delivery, particularly to a general formulation of nebulized inhaled lipid nanoparticles.

### BACKGROUND ART

Nucleic acid drugs achieve the purpose of preventing and treating diseases by regulating the expression of target genes. Nucleic acid drugs are of high specificity and high efficiency, and show promise in overcoming the drug development limitations of existing targets, with great potential for treating “non-targetable” and “non-druggable” diseases. However, in order to realize the enormous potential of nucleic acid drugs, efficient and safe delivery vector technologies are essential, especially for solving the key technical challenges of organ/cell-specific delivery of nucleic acid therapeutic molecules, and improving the stability as well as the bioavailability, and pharmacokinetic properties of nucleic acid drugs.

Currently, the most effective nucleic acid drug delivery vector is lipid nanoparticles (LNP), which is self-assembled from ionizable lipid compounds, neutral helper lipids, structural lipids, and PEG lipids. The ionizable lipid plays a key role, with its pKa generally between 5.5 and 6.8. This characteristic keeps its surface charge essentially neutral in the serum environment, facilitating cellular uptake of the entire LNP containing nucleic acid fragments to form endosomes. Once endocytosed by the cell, the acidic environment of the endosome protonates the head group of the ionizable lipid, inducing a cationic charge that destabilizes the endosomal membrane via electrostatic interactions. This results in payload (nucleic acid drug) release into the cytosol, enabling therapeutic activity through either protein expression or gene silencing.

Currently, several nucleic acid drugs utilizing lipid nanoparticles (LNPs) as delivery vehicles have been approved and marketed. These include Onpattro (which delivers small interfering RNA (siRNA)), LNPs administered via intravenous injection concentrating in the liver for RNA interference (RNAi) therapy, and mRNA vaccines administered via intramuscular injection to prevent infection by the coronavirus SARS-COV-2. Nucleic acid drugs are revolutionizing the development of next-generation therapeutics. In response to the global COVID-19 pandemic, there has been growing recognition of the importance and necessity for developing effective prevention and treatment strategies against respiratory diseases (particularly transformative nucleic acid-based therapies). However, due to the lack of lung tissue-specific delivery systems, no nucleic acid drugs capable of efficient pulmonary delivery have been currently approved for lung-related diseases, including pulmonary fibrosis, lung cancer, asthma, and bronchial disorders. Furthermore, studies have shown that for respiratory-transmitted diseases like COVID-19, adopting a virus-mimicking delivery route (i.e., specifically pulmonary inhalation) can induce systemic immunity (producing IgG antibodies) as well as trigger local mucosal immunity (generating IgA antibodies), resulting in combined cross-protection efficacy. Therefore, inhalable nucleic acid delivery vehicles hold particular significance for developing vaccines and therapeutics targeting respiratory diseases.

Inhalable nebulized formulations can directly deliver drugs to the lungs, and significantly enhancing drug enrichment in the lungs while reducing systemic toxicity, which is a unique advantage for respiratory delivery. However, the development of inhaled formulations, especially lipid nanoparticle (LNP)-based nucleic acid inhalable therapeutics, faces challenges related to poor nebulizability. These include nucleic acid leakage due to shear stress-induced damage during nebulization, increased particle size and polydispersity index (PDI) post-nebulization, and significantly reduced transfection efficiency after nebulization. Therefore, the primary key to developing aerosolized inhalable LNP nucleic acid drugs is to overcome the destructive effects of nebulization on LNP. All nebulization methods (including compressed air nebulization, ultrasonic nebulization, mesh nebulization, soft mist inhalers, and pressurized metered-dose inhalers (pMDIs)) subject LNPs to shear forces. This shear stress causes particle size enlargement, increased PDI, and decreased encapsulation efficiency (EE) due to nucleic acid leakage, ultimately impairing transfection efficacy. Currently, there are very few patents or studies focus on inhalable LNP-based nucleic acid drugs. Most reports optimize LNP component ratios to minimize changes in particle size, PDI, and EE before and after nebulization. However, even the best formulations identified show at least a doubling of particle size and halving of EE post-nebulization, yielding suboptimal results. Ensuring LNP structural integrity and stability under shear stress during nebulization and maintaining consistency pre- and post-nebulization is a critical technical challenge urgently needing resolution in the field.

The present invention provides a general formulation that enables the prepared LNPs to resist nebulization shear forces, ensuring consistent particle size, polydispersity index (PDI), and encapsulation efficiency (EE) before and after nebulization. This innovation overcomes the technical barriers of existing market nebulized LNP formulations that lack shear resistance and exhibit poor stability. This formulation is versatile and applicable to various types and component ratios of LNPs. Validation through in vivo mouse imaging experiments demonstrates that LNPs formulated with this method achieve effective pulmonary deposition compared to conventional LNPs, enabling gene transfection efficiency in the lungs to increase by several orders of magnitude.

### SUMMARY OF THE INVENTION

To address the deficiencies in the existing technology, the purpose of this invention is to provide a general formulation for nebulization inhalation of lipid nanoparticles (LNP), enabling LNP to withstand the nebulization shear forces during the nebulization process, improving the stability of LNP during nebulization, and achieving efficient delivery.

To achieve the above objectives, the present invention adopts the following technical solution:

A general formulation of nebulized inhaled lipid nanoparticles, wherein comprises a surface tension-lowering regulator added into a lipid nanoparticle dispersion system to increase the nebulization shear resisting ability of lipid nanoparticles. It should be noted that: any compound or composition that can be added to the LNP dispersion system to increase LNP's resistance to nebulization shear and improve the consistency of particle size, PDI, and EE before and after nebulization falls within the scope of protection of this invention and is inspired by this invention.

The general formulation of nebulized inhaled lipid nanoparticles of LNP, as a preferred embodiment, includes: an LNP dispersion system and a surface tension-lowering regulator added to said LNP dispersion system; the particle size range of said LNP is 70~160 nm; the mass volume percentage range of said surface tension reducing regulator in the LNP dispersion system is 0.01%~30%, for example, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%; it should be noted that: LNP with particle size distribution between 70~160 nm can better withstand the destructive effects of the nebulization process.

The general formulation of nebulized inhaled lipid nanoparticles of LNP, wherein the LNP dispersion system includes: LNP, and a system solution; the mass volume percentage range of the LNP in the dispersion system is 0.0025%~10%, and the concentration range of said system solution is 0~1000 mM.

The general formulation of nebulized inhaled lipid nanoparticles of LNP, wherein the LNP dispersion system includes: LNP, system solution, and cryopreservation protectant; the mass volume percentage range of said LNP in its dispersion system is 0.0025%~10%, the concentration range of said system solution is 0~1000 mM, and the mass volume percentage concentration range of said cryopreservation protectant is 0%~20%; it should be noted that: the LNP dispersion system may or may not include a cryopreservation protectant.

In another preferred embodiment, the LNP includes: ionizable lipid:helper lipid:structural lipid:polymer-conjugated lipid=30~60:5~40:20~40:0.1~15, by molar ratio. More preferably, the LNP includes: ionizable lipid:helper lipid:structural lipid:polymer-conjugated lipid=40~60:5~30:25~40:1~10, by molar ratio. It should be noted that: the nanoparticles of this invention can pass through biological barriers and intracellular barriers faced in pulmonary delivery so as to accumulate in large amounts in the lungs. The content of polymer-conjugated lipid has better research value for LNP penetration into lung cells, and research based on this invention's formulation is also inspired by this invention. Biological barriers refer to: mucosal layer of tracheal and alveolar epithelial cells, mucociliary structures, and surface-active substances (e.g., pulmonary surfactant) of tracheal and alveolar epithelial cells; intracellular barriers refer to challenges in cellular uptake and insufficient lysosomal escape caused by the high negative charge and large molecular weight of nucleic acids themselves.

In another preferred embodiment, the system solution comprises: one or a combination of normal saline, 4-(2-hydroxyethyl) piperazine-1-ethanesulfonic acid buffer (HEPES), tris(hydroxymethyl)aminomethane buffer (Tris), Tris-EDTA buffer, phosphate buffer (PB) and phosphate-buffered saline (PBS) buffer, Dulbecco's phosphate-buffered saline (DPBS), citrate buffer, sulfate buffer, carbonate buffer, acetate buffer, Tris-buffered saline with Tween (TBST), EDTA containing buffer and its sodium salt, and the combinations of one or more of the above; it should be noted that the system solutions mentioned here are not exhaustive, but merely preferred options, and any solution that regulates or buffers the osmotic pressure or pH of the system falls within the scope of protection of this invention.

In another preferred embodiment, the surface tension-lowering regulator comprises: one or a mixture of several of ethanol, propylene glycol, benzyl alcohol, poloxamer 188, Tween-80 (or Polysorbate 80), glycerol, and polyoxyethylene-15-hydroxystearate; the above is not exhaustive, and any known or unknown surface tension reducing regulator that can reduce the surface tension of the dispersion system falls within the scope of protection of this invention.

In another preferred embodiment, the surface tension-lowering regulator is ethanol, and the mass volume percentage range of ethanol in the LNP dispersion system is 0.05~30%, for example, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%.

In another preferred embodiment, the surface tension-lowering regulator is propylene glycol, and the mass volume percentage range of propylene glycol in the LNP dispersion system is 0.05~30%, for example, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%.

In another preferred embodiment, the surface tension-lowering regulator is a composition of propylene glycol and ethanol, where the mass volume percentage range of propylene glycol in the LNP dispersion system is 0.05~30%, for example, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%, and the mass volume percentage range of ethanol in the LNP dispersion system is 0.05~30%, for example, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%.

In another preferred embodiment, the surface tension-lowering regulator is poloxamer 188, and the concentration range of poloxamer 188 in the LNP dispersion system is 0.5~10 mg/mL, such as 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL.

In another preferred embodiment, the surface tension-lowering regulator is a mixture of poloxamer 188 and ethanol, where the concentration range of poloxamer 188 in the LNP dispersion system is 0.5~10 mg/mL, and the mass volume percentage range of ethanol in the LNP dispersion system is 0.05~30%, for example, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%.

In another preferred embodiment, the surface tension-lowering regulator is Tween-80, and the volume percentage range of Tween-80 in the LNP dispersion system is 0.01~2%, for example, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%.

In another preferred embodiment, the surface tension-lowering regulator is a mixture of Tween-80 and ethanol, where the volume percentage range of Tween-80 in the LNP dispersion system is 0.01~2%, for example, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, and the mass volume percentage range of ethanol in the LNP dispersion system is 0.05~30%, for example, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, or 30%.

In another preferred embodiment, the surface tension-lowering regulator is polyoxyethylene-15-hydroxystearate, and the concentration range of polyoxyethylene-15-hydroxystearate in the lipid nanoparticle dispersion system is 0.05-20 mg/ml, preferably 0.1-10 mg/ml, more preferably 3-7.5 mg/mL, such as 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 7.5 mg/mL, 8 mg/mL, 9 mg/mL, 10 mg/mL.

The advantages of this invention include:

1. The inventors introduce molecules capable of reducing the surface tension of water into the LNP dispersion system, and unexpectedly found that LNPs prepared using this formulation demonstrate resistance to shear forces during nebulization, maintaining consistent particle size and polydispersity index (PDI) before and after nebulization. This invention overcomes the technical limitations of commercial nebulized LNPs, which exhibit poor shear resistance and instability, achieving unprecedented technical effects.

2. By introducing surface tension-reducing molecules into the LNP dispersion system, the inventors unexpectedly mitigated nucleic acid leakage during nebulization, ensuring near-complete preservation of encapsulation efficiency (EE) in LNPs before and after nebulization.

3. By introducing molecules that can reduce the surface tension of water into the LNP dispersion system, the inventors further unexpectedly achieve high pulmonary deposition and enhanced gene transfection efficacy in the lungs for LNPs prepared with surface tension-reducing molecules when evaluated before and after nebulization.

4. The formulation exhibits exceptional versatility, maintaining shear resistance during nebulization and preserving particle size, PDI, and EE consistency across diverse LNP lipid compositions and ratios.

5. Studies on surface tension-reducing molecules and LNP nebulization stability reveal that ethanol, as a surface tension-modulating agent, significantly enhances the stability of LNP particle size, PDI, and EE before and after nebulization.

6. Investigations demonstrate that combining poloxamer 188 with ethanol as surface tension-modulating agents produces synergistic improvements in stabilizing LNP particle size, PDI, and EE during nebulization.

7. Study of surface tension of water and LNP nebulization stability shows that co-formulating propylene glycol with ethanol as surface tension-modulating agents exhibits synergistic effects in enhancing LNP stability (particle size, PDI, and EE) throughout nebulization.

8. In vivo mouse imaging confirms that LNPs formulated with this method achieve efficient pulmonary deposition, enhance lung gene transfection efficiency by several orders of magnitude compared to conventional LNPs.

### BRIEF DESCRIPTION OF THE FIGURES

FIG. 1 is a schematic diagram of the particle size variations in LNPs within dispersion systems containing different ethanol concentrations, comparing their states before and after nebulization in example 1 of the present invention.

FIG. 2 is a schematic diagram of the particle size variations in LNPs with varying particle sizes within dispersion systems containing a fixed ethanol concentration, comparing their states before and after nebulization in example 1 of the present invention.

FIG. 3 is a schematic diagram of the particle size changes before and after nebulization of LNPs with different LNP concentrations within a 7% ethanol dispersion system in example 2.

FIG. 4 shows the imaging results of C12 LNP nebulization in mice using dispersion systems with and without 8% ethanol in example 3. FIG. 5 shows the particle size distributions of various LNPs in dispersion systems containing different ethanol concentrations before and after nebulization in example 4.

FIG. 6 shows the particle size variations of LNPs in dispersion systems containing either 2 mg/mL poloxamer-188 alone or a combination of 2 mg/ml poloxamer-188 and 4% ethanol during nebulization in example 4.

FIG. 7 shows the particle size profiles of LNPs in dispersion systems with varying concentrations of Tween-80, illustrating the effects on particle size before and after nebulization in example 4.

### DEFINITION OF TECHNICAL TERMS

As used herein, the term “surface tension-lowering regulator” refers to substances added to LNP dispersion systems to enhance the nebulization shear tolerance of LNPs, typically chemical substances that can reduce the surface tension of water upon addition, but not limited to such substances. These agents typically reduce surface tension of water upon addition, but are not limited to such compounds. Examples include common surfactants (e.g., anionic, cationic, zwitterionic, and nonionic surfactants), exemplified by poloxamers, polysorbates (Tween), and benzalkonium derivatives, or other agents such as ethanol, propylene glycol, phenethyl alcohol, glycerin, and polyoxy-15-hydroxystearate. It should be noted that any compound or composition that is added to the LNP dispersion system to increase LNP's resistance to nebulization shear and improve the consistency of particle size, PDI, and EE before and after nebulization falls within the scope of protection of this invention and is inspired by this invention.

The mass volume percentage and mass volume percentage concentration mentioned in this document all represent w/v %, i.e., 1% w/v means 1 g/100 ml mass/volume (w: mass, v: volume).

Nucleic acid(s) refers to deoxyribonucleic acid (DNA) and ribonucleic acid (RNA), which are biological macromolecules composed of multiple nucleotide monomers; nucleic acids are composed of nucleotides, and nucleotide monomers are composed of a pentose sugar, a phosphate group, a nitrogenous base, and optional modifications. Polymers containing ribose sugar are RNA, while those with deoxyribose sugar are DNA.

Drugs to be delivered include: nucleic acid molecules, small molecule compounds, peptides, proteins, and other pharmaceutically active agents.

Nucleic acid molecules include single-stranded DNA, double-stranded DNA, short isomers, mRNA, tRNA, rRNA, long non-coding RNA (lncRNA), microRNA (miRNA), small interfering RNA (siRNA), Telomerase RNA Component, small RNA (snRNA and scRNA), circular RNA (circRNA), synthetic miRNA (miRNA mimics, miRNA agomir, miRNA antagomir), antisense DNA, antisense RNA, ribozyme, asymmetric interfering RNA (aiRNA), Dicer-substrate RNA (dsRNA), small hairpin RNA (shRNA), transfer RNA (tRNA), messenger RNA (mRNA), gRNA, sgRNA, crRNA or tracrRNA, locked nucleic acid (LNA), peptide nucleic acid (PNA), morpholino antisense oligonucleotides, morpholino oligonucleotides, or custom biological oligonucleotides, etc., or combinations thereof. The examples here are not exhaustive and anything polymerized from nucleotide monomers can be used in this invention. mRNA, messenger RNA, of which the name is messenger ribonucleic acid, is a single-stranded ribonucleic acid synthesized via transcription from one DNA template strand which carrying genetic information to direct protein synthesis. mRNA can be classified as monocistronic mRNA (encoding a single gene) or polycistronic mRNA (encoding multiple genes). Additionally, mRNA may incorporate one or more functional nucleotide analogs, such as pseudouridine, 1-methylpseudouridine, or 5-methylcytosine. The examples provided are not exhaustive; any modified mRNA or derivative thereof may be employed in the present invention.

Small interfering RNA (siRNA), also called short interfering RNA or silencing RNA, is a class of double-stranded RNA molecules. siRNA is generated when dsRNA is cleaved by RNase III enzymes (e.g., Dicer) into double-stranded fragments of ~20-30 base pairs (bp) within cells. These dsRNA molecules can originate from exogenous sources (e.g., viral RNA replication intermediates or artificially introduced dsRNA) or endogenous sources (e.g., dsRNA formed by RNA-dependent RNA polymerase acting on single-stranded RNA in cells). siRNA is a short dsRNA with phosphorylated 5′ ends and hydroxylated 3′ ends with two protruding nucleotides. The guide strand complements the target mRNA and guides the RISC complex to bind to the mRNA to induce mRNA degradation. As siRNA can be rationally designed to silence any gene, it serves as a critical tool for post-genomic era applications, including gene function validation and drug targeting. This technology marks the advent of a transformative era in biomedicine.

Small molecule compounds may serve as active ingredients in therapeutic or prophylactic agents, such as anti-tumor drugs, anti-infective drugs, local anesthetics, antidepressants, anticonvulsants, antibiotics/antimicrobials, antifungals, anti-parasitics, hormones, hormone antagonists, immunomodulators, neurotransmitter antagonists, anti-glaucoma agents, anesthetics, or imaging agents, etc. These examples are not exhaustive.

Peptides are compounds formed by α-amino acids connected by peptide bonds, which are intermediate products of protein hydrolysis.

Proteins are biomacromolecules structured into specific spatial conformations through dehydration condensation of amino acids into polypeptide chains. Examples include interferons, protein hormones, cytokines, chemokines, or enzymes.

Diluents refer to any pharmaceutically acceptable water-soluble excipients known in the art, including: amino acids, monosaccharides, disaccharides, tris-saccharides, tetra-saccharides, penta-saccharides, other oligosaccharides, mannitol, dextran, sodium chloride, sorbitol, polyethylene glycol (PEG), phosphates, their derivatives, or combinations thereof.

Charged lipid compounds refer to a class of lipid compounds that existing in a positively or negatively charged forms. Their charge is independent of physiological pH ranges (e.g., pH 3-9) and remains unaffected by pH variations. Charged lipids may be synthetic or naturally derived. Examples of charged lipids include, but are not limited to, DOTAP, DOTMA, and 18PA. These examples are not exhaustive. Any LNP composition utilizing the formulations of the present invention falls within the scope of protection and is encompassed by the teachings herein.

Helper lipids include phosphatidylcholine, phosphatidylethanolamine, sphingomyelin (SM), sterols and their derivatives, ceramides, or combinations of one or several charged lipids. Preferred examples of phosphatidylcholine include one or more of DSPC, DPPC, DMPC, DOPC, and POPC. Preferred examples of phosphatidylethanolamine include DOPE. Preferred sterols include cholesterol. These examples are not exhaustive. Any LNP composition utilizing the formulations of the present invention falls within the scope of protection and is encompassed by the teachings herein.

Structural lipids include one or more of cholesterol, non-sterols, sitosterol, ergosterol, campesterol, stigmasterol, brassicasterol, tomatine, tomatidine, ursolic acid, α-tocopherol, or corticosteroids. These examples are not exhaustive, and the selection of structural lipids is not limited. Polymer-conjugated lipid refer to PEGylated lipids. Exemplary polymer-conjugated lipids include, but are not limited to, one or more of PEG-modified phosphatidylethanolamine, PEG-modified phosphatidic acid, PEG-modified ceramide, PEG-modified dialkylamine, PEG-modified diacylglycerol, or PEG-modified dialkylglycerol. These examples are not exhaustive, and the selection of polymer-conjugated lipids is unrestricted.

DSPC: English name: distearoyl Phosphatidylcholine, 1,2-distearoyl-sn-glycero-3-phosphocholine; Chinese name: [symbol] , CAS No.: 816-94-4.

DPPC: Chinese name: [symbol] [symbol] ; English name: 1,2-dipalmitoyl-sn-glycero-3-phosphocholine, CAS No.: 63-89-8.

DMPC: Chinese name: [symbol] [symbol] , English name: 1,2-dimyristoyl-sn-glycero-3-phosphocholine, CAS No.: 18194-24-6.

DOPC: Chinese name: 1,2- [symbol] -sn- [symbol] -3- [symbol] ; English name: 1,2-dioleoyl-sn-glycero-3-phosphocholine, CAS No.: 4235-95-4.

POPC: Chinese name: 2- [symbol] -1- [symbol] -3- [symbol] ; English name: 2-oleoyl-1-palmitoyl-sn-glycero-3-phosphocholine, CAS No.: 26853-31-6.

DOPE: Chinese name: 1,2- [symbol] -3-SN- [symbol] -3- [symbol] ; English name: 1,2-dioleoyl-sn-glycero-3-phosphoethanolamine, CAS No.: 4004 May 1.

DOTAP: Chinese name: 1,2- [symbol] -3- [symbol] - [symbol] ); English name: 1,2-dioleoyl-3-trimethylammonium-propane (chloride salt), CAS No.: 132172-61-3; of which the chemical structure is shown below:

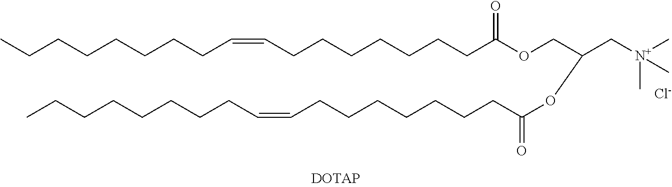 [SMILES: CCCCCCCC/C=C\CCCCCCCC(=O)OCC(C[N+](C)(C)C)OC(=O)CCCCCCC/C=C\CCCCCCCC ; [Cl-]]

DOTMA: name: N,N,N-Trimethyl-2,3-bis(octadec-9-en-1-yloxy) propan-1-ium chloride, CAS No.: 1325214-86-5, of which the chemical structure is shown below:

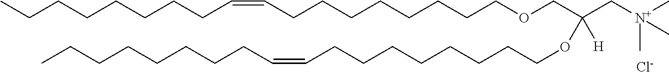 [SMILES: CCCCCCCC/C=C\CCCCCCCCOCC(C[N+](C)(C)C)OCCCCCCCC/C=C\CCCCCCCC ; [Cl-]]

18PA: CAS No.: 108392-02-5, of which the chemical structure is shown below:

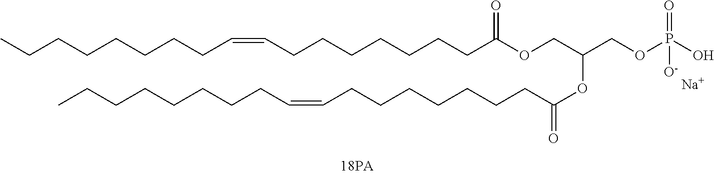 [SMILES: CCCCCCCC/C=C\CCCCCCCC(=O)OCC(COP(=O)([O-])O)OC(=O)CCCCCCC/C=C\CCCCCCCC ; [Na+]]

SM: Chinese name: [symbol] (SM); English name: sphingomyelin.

PEG: Chinese name: [symbol] ; English name: Polyethylene glycol.

Buffer solution: a buffer solution refers to a mixed solution composed of a weak acid and the salt thereof, or a weak base and the salt thereof. It can counteract or mitigate the impact of externally added strong acids or bases on the solution's pH, thereby maintaining a relatively stable pH value. Examples include phosphoric acid, citric acid, carbonic acid, acetic acid, sodium acetate, ammonium acetate, barbituric acid, Tris, PBS, HEPES, EDTA and its sodium salts, or combinations thereof. These examples are not exhaustive.

HEPEs buffer: Chinese name: 4- [symbol] [symbol] ; English name: 2-[4-(2-hydroxyethyl) piperazin-1-yl]ethanesulfonic acid; CAS No.: 7365-45-9.

Tris buffer: name: 2-Amino-2-(hydroxymethyl)-1,3-propanediol; 2-Amino-2-(hydroxymethyl)-1,3-propanediol; 2-Amino-2-(hydroxymethyl)-1,3-propanediol; 2-Amino-2-(hydroxymethyl) propane-1,3-diol; CAS No.: 77-86-1.

Citrate buffer: The main components of citrate buffer are citric acid and disodium hydrogen phosphate.

DPBS: Dulbecco's phosphate-buffered saline.

TBST: Composed of Tris-HCl, NaCl, tween20.

EDTA sodium salt: EDTA disodium dihydrate is ethylenediaminetetraacetic acid disodium dihydrate, English name: EDTA disodium salt dihydrate.

PBS buffer: The main components are Na₂HPO₄, KH₂PO₄, NaCl, and KCl.

Cryoprotectant: a cryoprotectant refers to substances (usually solutions) that protect cells from cryoinjury during freezing. They can be categorized into: sugars, alcohols, amino acids, salts, etc. Specific examples include sucrose, mannitol, trehalose, lactose, glucose, maltose, polyvinylpyrrolidone (PVP), polyethylene glycol (PEG), dextran, albumin, and hydroxyethyl starch. These examples are not exhaustive.

Unless otherwise specified, the terms “particle size” and “hydrodynamic diameter”, “hydrodynamic particle size” can be used interchangeably in this application, all referring to hydrodynamic diameter.

### EMBODIMENTS

The following provides a specific introduction to this invention in conjunction with the drawings and specific embodiments.

This invention verifies the technical effect of the universal formulation through the following experiments:

### Experimental Materials:

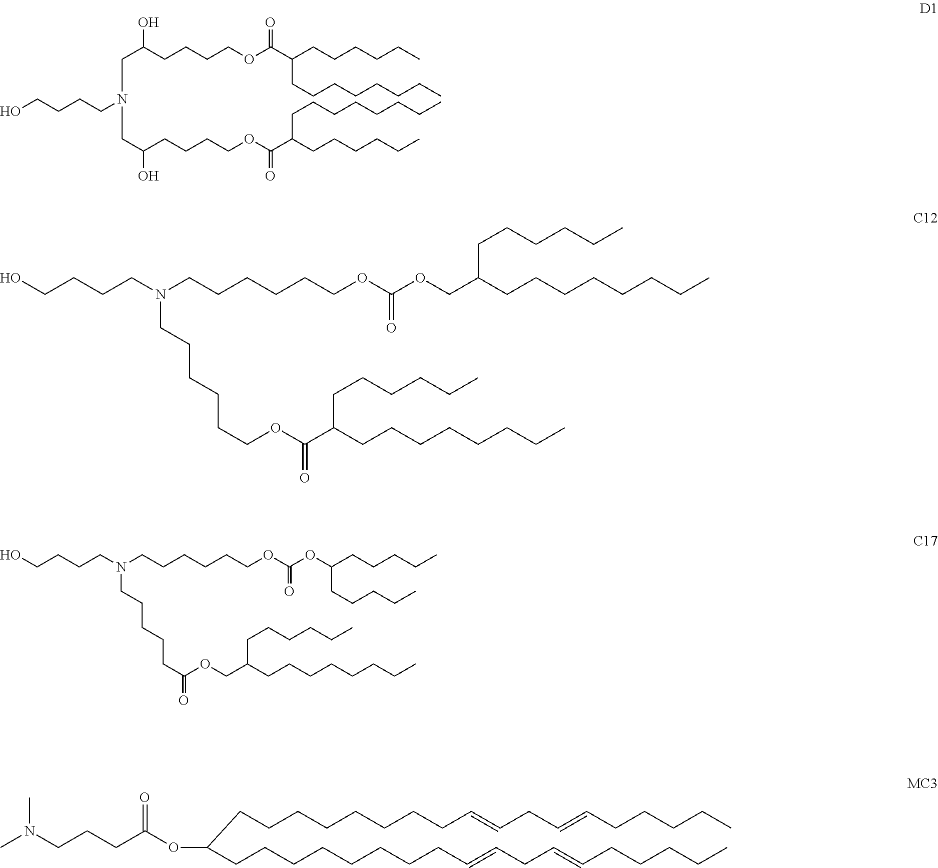

### Example 1: Impact of Ethanol as a Surface Tension-Reducing Agent on Particle Size, Polydispersity Index (PDI), and Encapsulation Efficiency (EE) of Lipid Nanoparticles (LNP) Before and After Nebulization

1.1 Changes in Dispersed Systems on LNP Particle Size, PDI, and EE Before And after Nebulization in Different Ethanol Contents:

LNP Formulation of Example 1 (molar ratio): The molar ratio of components of lipid for Example 1 LNPs was C12: DSPC: cholesterol: DMG-PEG=46.3:9.4:42.7:1.6.

Experimental method: Example 1 LNPs were fabricated using a microfluidic device. Ethanol (absolute) was used to dissolve the lipid mixture, while mRNA was diluted in sodium acetate buffer. The aqueous-to-ethanol phase ratio was maintained at 1:3. Flow rates were optimized to achieve LNP particle sizes between 110-140 nm. After preparation, LNPs were incubated for 20 minutes, then dialyzed in 20 mM HEPES buffer (pH 6.0) containing 10% sucrose. Aliquots (50 μL) were 3-fold diluted with buffer containing varying ethanol concentrations. Nebulization was performed using a vibrating mesh nebulizer (Aerogen Solo Nebulizer System). Particle size, PDI, and EE were measured before and after nebulization.

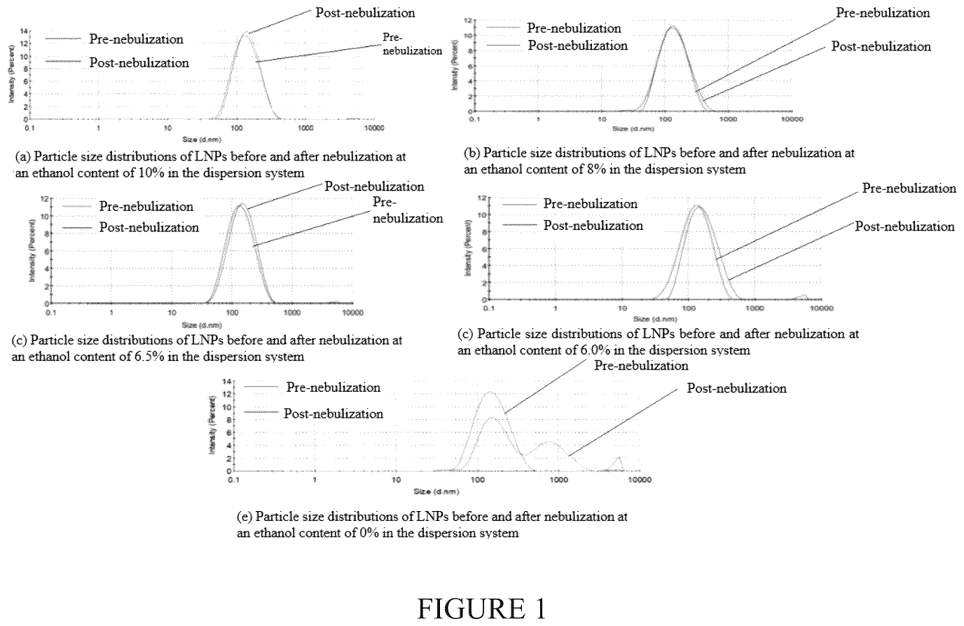

Experimental results were shown in Table 1 and FIG. 1:

TABLE 1
Changes in particle size, PDI, and EE of LNP before and after nebulization
in dispersion systems with different ethanol contents

| | | Ethanol | Pre-/post- | | |
|---|---|---|---|---|---|
| | Ethanol | concentration | nebulization | Pre-/post- | |
| | concentration | in dispersion | particle size | nebulization | Pre-/post- |
| No. | in diluent | system | (nm) | PDI | nebulization EE |
| 1 | 40% | 30% | 125.3/125.5 | 0.160/0.158 | 94.8/94.6 |
| 2 | 15% | 10% | 127.6/133.1 | 0.151/0.143 | 95.7/93.4% |
| 3 | 12% | 8% | 114.4/124.3 | 0.190/0.179 | 94.6%/93.5% |
| 4 | 11.25% | 7.5% | 116.5/126.4 | 0.191/0.180 | 93.6%/93.2% |
| 5 | 10.5% | 7% | 115.7/124.8 | 0.201/0.190 | 94.5%/94.1% |
| 6 | 9.75% | 6.5% | 120.4/132.3 | 0.215/0.189 | 93.7%/93.0% |
| 7 | 9% | 6% | 113.1/145.2 | 0.188/0.214 | 94.2%/84.5% |
| 8 | 0.1% | 0.05% | 110.1/143.5 | 0.165/0.201 | 96.4%/90.3 |
| 9 | 0 | 0 | 138.7/260.6 | 0.162/0.504 | 93.5%/70.2% |

Experimental conclusion: For the LNPs in Example 1, if the dispersion system does not contain ethanol, the particle size nearly doubles after nebulization, PDI increased to above 0.5, and EE decreased significantly. As long as ethanol was included in the system, the destructive effects of nebulization were effectively mitigated, ensuring minimal changes in particle size, PDI, and EE before and after nebulization, and with increasing ethanol content, the changes were even smaller. For this formulation, ethanol concentrations between 0.01% to 30% (v/v) in the dispersion system were suitable.

1.2 Optimization of Applicable Particle Size Range for Example 1 LNPs with Fixed Ethanol Content (6.5%) to Investigate the Particle Size Range where No Significant Changes in Particle Size, PDI, or EE Occurred Before and After Nebulization.

Experimental Method: Lipid nanoparticles (LNPs) of Example 1 were fabricated using a microfluidic device. The lipid formulation of Example 1 LNPs was dissolved in anhydrous ethanol, while mRNA was diluted in sodium acetate buffer. The ethanol-water phase ratio was maintained at 1:3, and flow rates were adjusted to prepare LNPs with varying particle sizes. After preparation, LNPs were incubated for 20 min, followed by dialysis in 20 mM HEPES buffer (pH 6) containing 10% sucrose. Aliquots (50 μL) were then 3-fold diluted with ethanol-containing buffer, nebulized, and analyzed for particle size, PDI, and EE before and after nebulization.

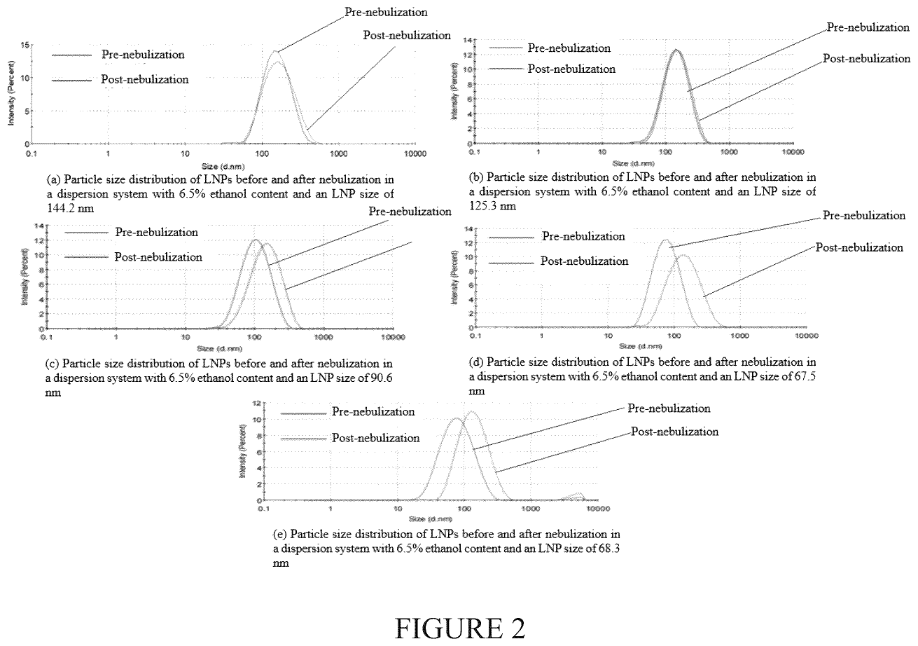

Experimental results were shown in Table 2 and FIG. 2:

TABLE 2
the changes in particle size, polydispersity index (PDI),
and encapsulation efficiency (EE) of LNPs with varying
particle sizes in dispersion systems with fixed ethanol
concentrations before and after nebulization

| | | Pre-/post- | | |
|---|---|---|---|---|
| | | nebulization | Pre-/post- | Pre-/post- |
| LNP | Flow rate | particle size | nebulization | nebulization |
| No. | mL/min | (nm) | PDI | EE |
| 1 | 1 | 160.0/165.2 | 0.151/0.165 | 96.8%/92.3% |
| 2 | 1.6 | 144.2/151.3 | 0.136/0.154 | 97.4%/94.8% |
| 3 | 1.8 | 132.4/132.5 | 0.151/0.151 | 96.7%/94.3% |
| 4 | 2 | 125.3/136.9 | 0.167/0.159 | 95.0%/94.2% |
| 5 | 2.4 | 103.1/105.8 | 0.147/0.138 | 95.5%/92.4% |
| 6 | 4 | 90.6/127.1 | 0.173/0.189 | 97.1%/91.7% |
| 7 | 5 | 80.0/100.7 | 0.165/0.187 | 96.8/92.4 |
| 8 | 6 | 70.2/90.3 | 0.159/0.188 | 94.9%/90.7% |
| 9 | 8 | 65.3/125.3 | 0.165/0.349 | 93.9%/83.1% |

Experimental conclusion: LNPs of different particle sizes exhibited variations in particle size, PDI, and EE before and after nebulization in a 6.5% ethanol-containing HEPES dispersion system, wherein LNPs with particle sizes between 70-160 nm showed minimal parameter fluctuations. When particle sizes were smaller than 70 nm, for example at 65.3 nm, the LNP particle sizes nearly doubled in size after nebulization, with PDI increased to 0.349 and a significant decrease in EE. Therefore, controlling LNP particle sizes within 70-160 nm ensured better post-nebulization stability.

### 1.3 Study on Changes in Particle Size, PDI, and EE of LNPs of Different Concentrations Before and After Nebulization (Fixed Ethanol Content)

Experimental method: Lipid nanoparticles (LNPs) of Example 1 were fabricated using a microfluidic device. The lipid formulation of Example 1 LNPs was dissolved in anhydrous ethanol, while mRNA was diluted in sodium acetate buffer. The ethanol-water phase ratio was maintained at 1:3, and flow rates were adjusted to prepare LNPs with particle sizes distributed between 70-160 nm. After preparation, LNPs were incubated for 20 minutes, then dialyzed in 20 mM HEPES buffer (pH 6) containing 10% sucrose and 7% ethanol. Aliquots (150 μL) were either nebulized directly or diluted with 7% ethanol-containing buffer at different ratios before nebulization. Particle size, PDI, and EE were measured before and after nebulization.

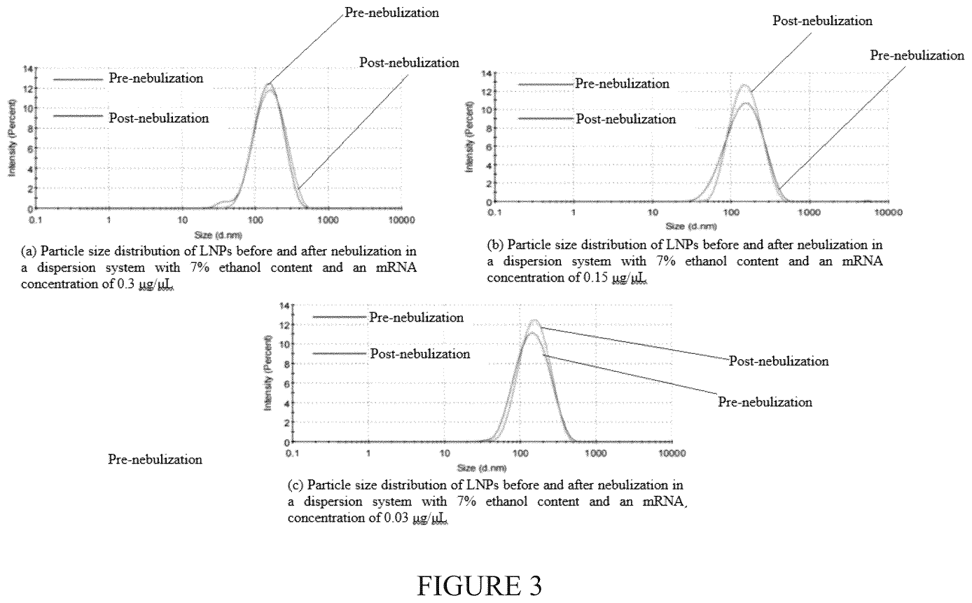

Experimental results were shown in Table 3 and FIG. 3:

TABLE 3
The particle size, polydispersity index (PDI), and encapsulation
efficiency (EE) of LNPs at varying concentrations in a 10%
ethanol dispersion system before and after nebulization

| | Concentration | Pre-/post- | Pre-/post- | Pre-/post- |
|---|---|---|---|---|
| Concentration of LNP | of mRNA | nebulization | nebulization | nebulization |
| (w/v)% | μg/μL | particle size (nm) | PDI | EE |
| 10 | 2.5 | 129.3/141.7 | 0.189/0.176 | 98.4/98.1 |
| 1 | 0.5 | 126.9/139.0 | 0.201/0.169 | 98.4/96.7 |
| 0.0025% | 0.005 | 127.7/137.0 | 0.194/0.165 | 98.4/97.5 |

Experimental conclusion: If diluted, nebulization would be more stable. It was equally stable at mRNA concentrations of 0.5 μg/μL and 0.005 μg/μL, where the mRNA concentration of 0.005 μg/μL corresponds to an LNP mass volume fraction of 0.0025%, i.e., nebulization was stable when the mass volume percentage of LNP in the dispersion system was 0.0025%~10%.

Experimental Conclusion: In a system with 7% ethanol content, nebulization was stable even when the mRNA concentration reached 2.5 μg/μL (corresponding to a lipid nanoparticle (LNP) mass/volume fraction of 10%), and was even more stable when diluted. LNP of mRNA concentration of 0.5 μg/μL and 0.005 μg/μL were of similar stability. The LNP mass/volume fraction at 0.005 μg/μL was 0.0025% (w/v), therefore, nebulization stability was maintained for LNP mass/volume fractions ranging from 0.0025% to 10% (w/v).

### Experiment 2: Experiment to Verify the Universality of the Formulation

By substituting components in the LNP composition while retaining the dispersion system with a surface tension-reducing agent, LNP nebulization tolerance was investigated to demonstrate the universality of the formulation.

Experimental method: Ionizable lipid compounds and formulations were modified as shown in Examples 2-4 LNPs. A commercial DLin-MC3-DMA (abbreviated as MC3) was selected as a comparative control. The molar ratios of the formulations were listed in Table 4. Microfluidic preparation parameters: front waste 0.3 mL, tail waste 0.05 mL, total volume 0.75 mL, total flow rate 2-8 mL/min, volume ratio: 3:1. LNPs were fabricated to achieve particle sizes between 70-160 nm, and dialyzed with HEPES buffer. LNP (50 μL) were collected, diluted with 100 μL ethanol-containing HEPES buffer to achieve varying ethanol concentrations, nebulized, and analyzed for particle size, PDI, and EE before and after nebulization.

TABLE 4
Formulation molar ratios for each LNP embodiment

| Name of LNP | Formulation (molar ratio) | | | |
|---|---|---|---|---|
| Example 1 LNP | D1 | DOPC | Cholesterol | DMG-PEG2000 |
| | 47.5 | 10 | 40.8 | 1.7 |
| Example 2 LNP | C17 | DSPC | Cholesterol | DMG-PEG2000 |
| | 46.3 | 9.4 | 42.7 | 1.6 |
| Example 3 LNP | C12 | DSPC | Cholesterol | DMG-PEG2000 |
| | 60 | 20 | 19 | 1 |
| Comparative | MC3 | DSPC | Cholesterol | DMG-PEG2000 |
| example LNP | 50 | 10 | 38.5 | 1.5 |

Experimental results were shown in Table 5 and FIG. 5:

TABLE 5
Particle size, PDI, and EE for each LNP in dispersion systems
with different ethanol contents before and after nebulization.

| | Ethanol | | | |
|---|---|---|---|---|
| | concentration | Pre-/post- | | |
| | in dispersion | nebulization | Pre-/post- | Pre-/post- |
| Name of LNP | system | particle size (nm) | nebulization PDI | nebulization EE |
| Example 1 | 6.5% ^{a} | 142.4/183.2 | 0.166/0.245 | ~ |
| LNP | | | | |
| Example 1 | 8% ^{a} | 141.9/178.7 | 0.173/0.211 | 94.5%/90.3% |
| LNP | | | | |
| Example 1 | 20% ^{a} | 142.0/146.2 | 0.165/0.165 | ~^{b} |
| LNP | | | | |
| Example 2 | 6.5% ^{a} | 135.0/154.7 | 0.155/0.185 | ~^{b} |
| LNP | | | | |
| Example 2 | 8% ^{a} | 134.7/141.3 | 0.166/0.169 | 96.9%/91.8% |
| LNP | | | | |
| Example 2 | 10% ^{a} | 133.5/139.4 | 0.170/0.171 | ~^{b} |
| LNP | | | | |
| Example 2 | 20% ^{a} | 135.1/137.2 | 0.161/0.161 | ~^{b} |
| LNP | | | | |
| Example 3 | 6.5% ^{a} | 134.7/143.2 | 0.221/0.202 | 95.4%/90.0% |
| LNP | | | | |
| Comparative | 25% ^{a} | 159.5/160.2 | 0.155/0.154 | 96.6%/93.5% |
| example LNP | | | | |
| Comparative | 10% ^{a} | 120.5/126.2 | 0.145/0.146 | 97.6%/93.5% |
| example LNP | | | | |
| Comparative | 5% ^{a} | 75.5/84.2 | 0.165/0.176 | 95.6%/91.5% |
| example LNP | | | | |

Note:
^{a} represents ethanol content in the dispersion system,
^{b} represents not tested.

Experimental conclusion: For different types of LNPs or different LNP formulations, as long as the particle size was within the range of 70~160 nm, consistent particle size, PDI, and EE before and after nebulization can be achieved by adjusting the ethanol content in the dispersion system. The required ethanol concentration varies by LNP type but generally does not exceed 30% (v/v).

### Experiment 3: Expression in the Lungs after Nebulization Inhalation of LNP in Mice

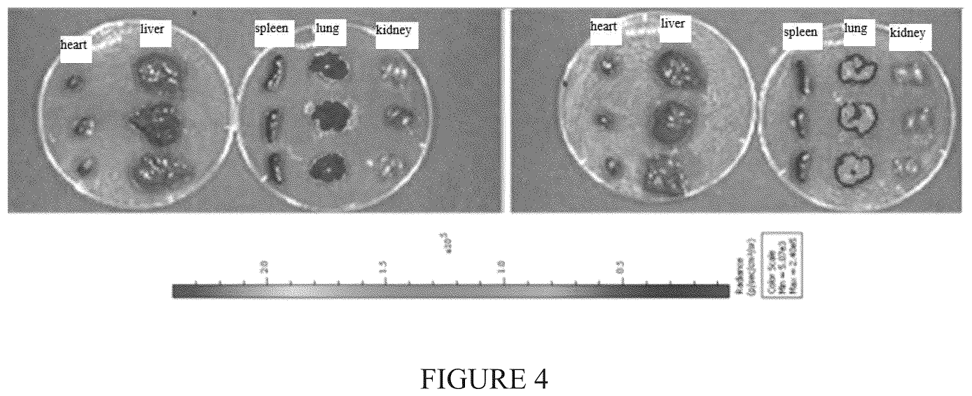

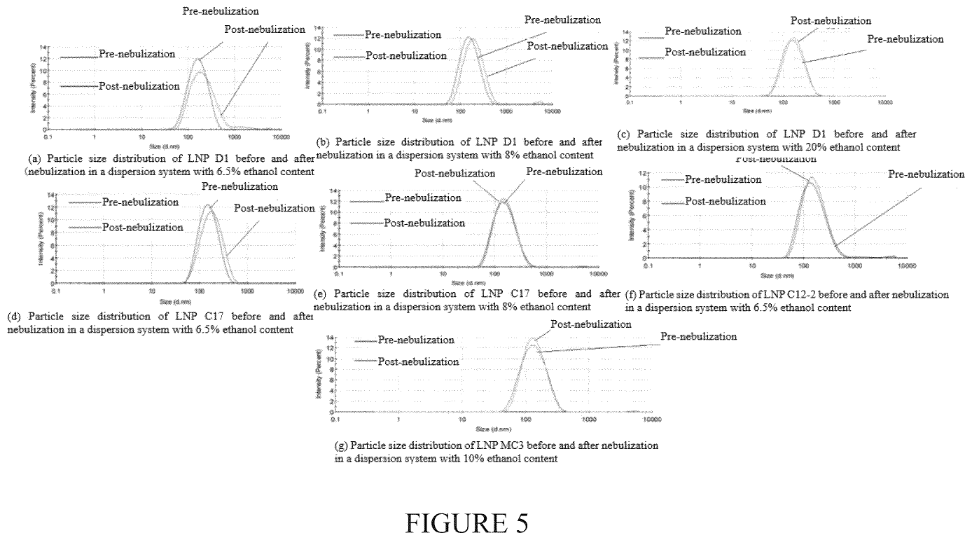

Experimental method: Example 1 LNPs loaded with Luciferase mRNA were fabricated via microfluidic methods. Mice were nebulize inhaled with the LNPs, intraperitoneally injected with fluorescein potassium substrate after 6 hours, and euthanized 5 minutes later. Organs (heart, liver, spleen, lung, kidney) were imaged (FIG. 4).

Experimental conclusion: LNPs nebulized in ethanol-containing buffers showed significantly higher transfection efficiency compared to ethanol-free buffers. Fluorescence quantification values for ethanol-buffered LNPs exceeded those of ethanol-free buffers by multiple orders of magnitude, demonstrating effective lung deposition and tolerance to shear stress during nebulization.

### Experiment 4: Impact of Alternative Surface Tension-Reducing Agents on LNP Particle Size and PDI Stability Before/after Nebulization

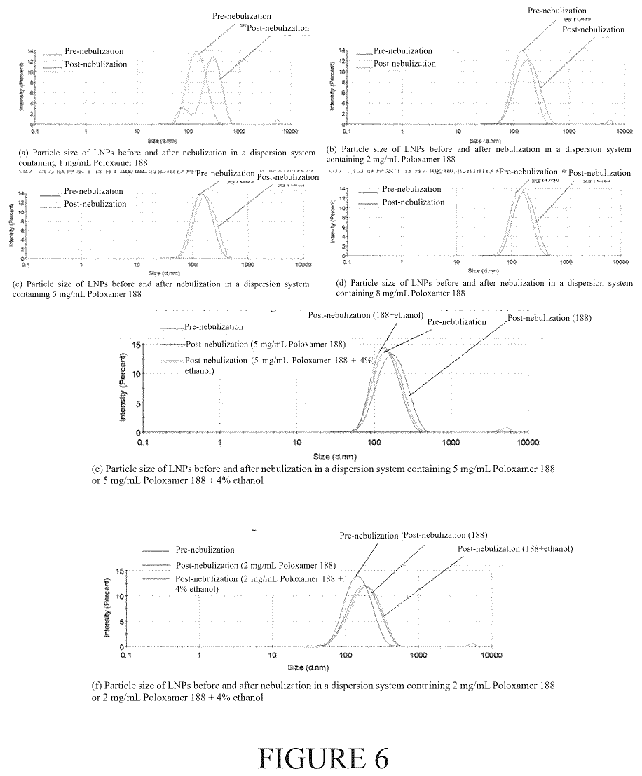

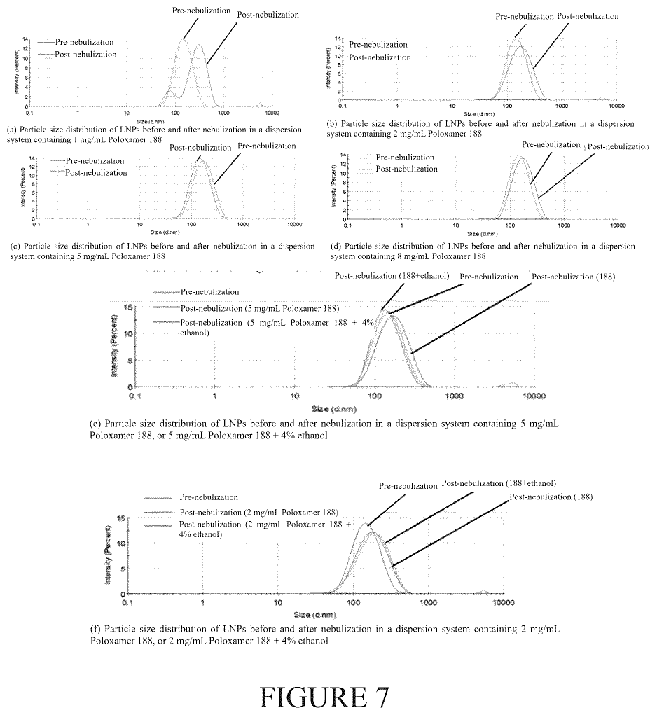

Experimental Method: Example 1 LNPs were fabricated using a microfluidic device. Lipid formulations were dissolved in anhydrous ethanol, mRNA was diluted in sodium acetate buffer, and the ethanol-water phase ratio maintained at 1:3. Flow rates were adjusted to produce LNPs with ~130 nm particle size. After preparation, LNPs were incubated for 20 minutes and dialyzed in 20 mM HEPES buffer (pH 6.0) containing 10% sucrose. Then, aliquots (50 μL) were separately diluted 3-fold with HEPEs buffer containing either one or two combinations of the following surface tension-reducing agents: Poloxamer 188, Tween-80, phenethyl alcohol, propylene glycol, glycerin, or ethanol. After nebulization, particle size and PDI before and after nebulization were tested. When the surface tension-reducing agent was Poloxamer 188 or ethanol, the results were shown in Table 6 and FIG. 6. When the surface tension-reducing agent was Tween-80 or ethanol, the results were shown in Table 7 and FIG. 7

TABLE 6
Changes in particle size and PDI of LNP when poloxamer 188 was
added to the dispersion system before and after nebulization

| | | Particle size | |
|---|---|---|---|
| | Final dispersion system of LNPs | (nm) | PDI |
| before | HEPEs buffer system | 133.6 | 0.152 |
| nebulization | | | |
| After | 0.4 mg/mL Poloxamer 188 | 246.4 | 0.261 |
| nebulization | 0.5 mg/mL Poloxamer 188 | 168.5 | 0.228 |
| | 2 mg/mL Poloxamer 188 | 169.1 | 0.225 |
| | 3 mg/mL Poloxamer 188 | 175.3 | 0.243 |
| | 5 mg/mL Poloxamer 188 | 153.8 | 0.164 |
| | 8 mg/mL Poloxamer 188 | 151.5 | 0.178 |
| | 10 mg/mL Poloxamer 188 | 151.4 | 0.175 |
| | 5 mg/mL Poloxamer 188 + 4% | 138.6 | 0.180 |
| | ethanol | | |
| | 2 mg/mL Poloxamer 188 + 4% | 161.7 | 0.175 |
| | ethanol | | |

Experimental conclusion: The introduction of the surface tension-reducing agent Poloxamer 188 enhanced nebulization stability. For C12-LNPs, when the surface tension-reducing agent was Poloxamer 188, no significant changes in particle size, PDI, or EE were observed within a concentration range of 0.5-10 mg/mL. When the concentration of poloxamer 188 was 5~10 mg/mL, the variations in LNP parameters before and after nebulization were minimized. Notably, when 4% ethanol was introduced into the system, stability was maintained at both 5 mg/mL and 2 mg/mL Poloxamer 188 concentrations. Therefore, the combination of ethanol and Poloxamer 188 exhibited an unexpected synergistic effect in improving the stability of particle size and PDI before and after nebulization.

TABLE 7
Changes in particle size and PDI of LNP when Tween-80 was added
to the dispersion system before and after nebulization

| | | Particle size | |
|---|---|---|---|
| | Final dispersion system of LNPs | (nm) | PDI |
| before | HEPEs buffer system | 117.2 | 0.176 |
| nebulization | | | |
| After | 2% v/v Tween-80 | 121.2 | 0.282 |
| nebulization | 1% v/v Tween-80 | 121.3 | 0.297 |
| | 0.5% v/v Tween-80 | 126.3 | 0.242 |
| | 0.25% v/v Tween-80 | 123.1 | 0.253 |
| | 0.2% v/v Tween-80 | 130.3 | 0.248 |
| | 0.1% v/v Tween-80 | 139.4 | 0.238 |
| | 0.01% v/v Tween-80 | 140.6 | 0.222 |

Experimental conclusion: Tween-80, when added as a surface tension-reducing agent to LNP dispersion systems, also stabilizes LNP particle size before and after nebulization. Within a Tween-80 volume concentration range of 0.01% to 2%, no significant increase in particle size was observed after nebulization. However, since Tween-80 inherently self-assembles into micelles, this may lead to elevated PDI values detected by the particle size analyzer, though the intrinsic PDI of LNPs remained nearly unchanged compared to pre-nebulization levels. Additionally, increasing Tween-80 content further reduced particle size increases during nebulization.

TABLE 8
Characterization of particle size, PDI, and EE in systems with
different concentrations of Tween-80 or combined use of Tween-80
and ethanol when preparing larger particle size LNP at a total
flow rate of 4 mL/min before and after nebulization

| | Dispersion | | | |
|---|---|---|---|---|
| LNP | composition and | | | |
| preparation | nebulization | Hydrodynamic | | |
| flow rate | conditions | diameter (nm) | PDI | EE |
| 4 mL/min | No additive - Pre- | 112.2 | 0.213 | 98.8% |
| | nebulization | | | |
| | 2% Tween 80 - Pre- | 106.7 | 0.222 | |
| | nebulization | | | |
| | 2% Tween 80 - | 112.2 | 0.220 | |
| | Post-nebulization | | | |
| | 0.25% Tween 80 - | 109.4 | 0.221 | |
| | Pre-nebulization | | | |
| | 0.25% Tween 80 - | 112.4 | 0.218 | 87.5% |
| | Post-nebulization | | | |
| | 2% Ethanol + | 109.5 | 0.211 | |
| | 0.25% Tween 80 - | | | |
| | Pre-nebulization | | | |
| | 2% Ethanol + | 112.8 | 0.202 | 88.4% |
| | 0.25% Tween 80 - | | | |
| | Post-nebulization | | | |
| | 2% Ethanol + 0.5% | 110.3 | 0.209 | |
| | Tween 80 - Pre- | | | |
| | nebulization | | | |
| | 2% Ethanol + 0.5% | 113 | 0.210 | 86.3% |
| | Tween 80 - Post- | | | |
| | nebulization | | | |

TABLE 9
Characterization of particle size, PDI, and EE in systems with
different concentrations of Tween-80 or combined use of Tween-80
and ethanol when preparing smaller particle size LNP at a total
flow rate of 12 mL/min before and after nebulization

| | Dispersion | | | |
|---|---|---|---|---|
| LNP | composition and | | | |
| preparation | nebulization | Hydrodynamic | | |
| flow rate | conditions | diameter (nm) | PDI | EE |
| 4 mL/min | No additive - Pre- | 64.5 | 0.204 | 99.1% |
| | nebulization | | | |
| | 2% Tween 80 - Pre- | 61.7 | 0.222 | |
| | nebulization | | | |
| | 2% Tween 80 - Post- | 71.2 | 0.274 | |
| | nebulization | | | |
| | 0.25% Tween 80 - | 62.8 | 0.185 | |
| | Pre-nebulization | | | |
| | 0.25% Tween 80 - | 78.7 | 0.251 | 82.4% |
| | Post-nebulization | | | |
| | 2% Ethanol + 0.25% | 62.9 | 0.180 | |
| | Tween 80 - Pre- | | | |
| | nebulization | | | |
| | 2% Ethanol + 0.25% | 80.0 | 0.257 | 82.4% |
| | Tween 80 - Post- | | | |
| | nebulization | | | |
| | 2% Ethanol + 0.5% | 63.8 | 0.205 | |
| | Tween 80 - Pre- | | | |
| | nebulization | | | |
| | 2% Ethanol + 0.5% | 72.1 | 0.250 | |
| | Tween 80 - Post- | | | |
| | nebulization | | | |

Experimental conclusion: For two LNPs with different particle sizes (e.g., 112.2 nm in Table 8 and 64.5 nm in Table 9 before nebulization), the addition of Tween-80 resulted in reduced particle size prior to nebulization. For the larger LNP (112.2 nm), changes after nebulization were less compared to the smaller LNP (64.5 nm). Therefore, LNPs with particle sizes between 70-160 nm exhibited better tolerance to the destructive effects of nebulization. Additionally, the combination of ethanol and Tween-80 further enhanced nebulization stability.

### Experiment 5: Investigation of the Impact of Surface Tension-Reducing Agents on Nebulization Stability in Sucrose-Free HEPES Dispersions

TABLE 10
Influence of various surface tension reducing regulators introduced in HEPEs buffer
without sucrose on LNP nebulization stability before and after nebulization

| | Diameter | | |
|---|---|---|---|
| | Pre-/post- | Pre-/post- | |
| | nebulization | nebulization | Pre-/post- |
| LNP dispersion system | (nm) | PDI | nebulization EE |
| LNP original Liquid | 110.6 | 0.153 | 96.8% |
| 8% ethanol | 111.8/113.8 | 0.154/0.14 | 94.92%/92.85% |
| 6% ethanol | 112.6/115.2 | 0.153/0.146 | 93.47%/90.14% |
| 4% ethanol | 114.1/117.2 | 0.156/0.167 | 94.04%/92.85% |
| 3% ethanol | 111.8/122.8 | 0.151/0.192 | 95.20%/93.27% |
| 2% ethanol | 112.5/127 | 0.157/0.206 | 95.10%/91.92% |
| Tween-80 0.25% | 110.6/130.5 | 0.174/0.252 | |
| Tween-80 0.15% | 111.6/123.5 | 0.151/0.212 | 84.85%/80.83% |
| 2% ethanol + 0.05% Tween80 | 113/125.4 | 0.154/0.240 | |
| 5% glycerol | 113/156.1 | 0.136/0.317 | 95.19%/93.66% |
| 2% glycerol | 112.5/166.5 | 0.148/0.372 | |
| 0.5% Poloxamer 188 | 110.8/122.3 | 0.150/0.211 | 96.57%/95.10% |
| 12.3 mg/mL phenylethanol | 109.8/120.2 | 0.164/0.238 | 95.95%/91.40% |
| 6.18 mg/mL phenylethanol | 111.7/127.4 | 0.149/0.233 | 96.55%/83.32% |
| 30% propane-1,2-diol | 135.5/138.2 | 0.155/0.157 | 96.20%/96.00% |
| 25% propane-1,2-diol | 121.3/127.4 | 0.165/0.171 | 95.51%/95.31% |
| 15% propane-1,2-diol | 120.1/131.6 | 0.167/0.163 | 95.91%/95.31% |
| 10% propane-1,2-diol | 118.6/135.7 | 0.169/0.174 | 95.99%/94.96% |
| 5% propane-1,2-diol | 119.3/147.2 | 0.156/0.246 | 96.17%/95.62% |
| 0.05% propane-1,2-diol | 110.5/150.8 | 0.145/0.228 | 95.82%/90.35% |
| 2% ethanol+8% propane-1,2-diol | 121.0/131.9 | 0.176/0.167 | 96.02%/95.28% |
| 2% ethanol+10% propane-1,2-diol | 117.5/119.5 | 0.175/0.145 | 96.17%/95.63% |

Experimental conclusion: 1) The use of surface tension-reducing agents other than ethanol, such as Poloxamer 188, Tween-80, propylene glycol, phenethyl alcohol, glycerin, or combinations of these agents, can also enhance the stability of LNP particle size and PDI before and after nebulization. When the mass/volume concentration of propylene glycol was within 0.05% to 30%, minimal changes in particle size, PDI, and EE were observed.

2) If the dispersion system does not contain sucrose, the viscosity of the dispersion system can be reduced, enabling consistent particle size and EE before and after nebulization at lower concentrations of surface tension-reducing agents.

### Experiment 6: Impact of Surface Tension-Reducing Agent HS15 on Nebulization Stability

### 1) Experimental Purpose

LNPs were fabricated by encapsulating luc mRNA using C12 as the ionizable lipid via microfluidic device to investigate the nebulization stability. The formulation includes Polyoxy-15-hydroxystearate (abbreviated as HS15, commercially available). The experimental procedure and methods were identical to those in Experiment 1.

### 2) Experimental Results

TABLE 11
Particle size and encapsulation efficiency
of LNP before and after nebulization

| | Particle size | | | |
|---|---|---|---|---|
| Sample name | (d · nm) | PDI | EE (%) | Particle size (d · nm) |
| HS15-0.1-pre | 96.94 | 0.1293 | 90.5 | 86.7 |
| HS15-0.1-post | 112.3 | 0.1801 | 91.6 | 82.8 |
| HS15-2-pre | 98.03 | 0.1533 | 90.8 | 88.3 |
| HS15-2-post | 117.7 | 0.1904 | 92.5 | 84.4 |
| HS15-5-pre | 95.54 | 0.1368 | 91.0 | 88.5 |
| HS15-5-post | 111.5 | 0.1861 | 89.6 | 82.9 |
| HS15-10-pre | 96.97 | 0.1419 | 88.6 | 87.5 |
| HS15-10-post | 110.3 | 0.1863 | 86.7 | 86.0 |
| HS15-0-pre | 97.38 | 0.1465 | 92.1 | 87.1 |
| HS15-0-post | 127 | 0.229 | 64.1 | 60.2 |

Note:
“pre” and “post” refer to pre-nebulization and post-nebulization samples, respectively. HS15 concentrations were 0.1 mg/mL, 2 mg/mL, 5 mg/mL, and 10 mg/mL, with a control group lacking HS15. Conclusion: If the dispersion system does not contain HS15, the particle size significantly increases before and after nebulization, with PDI, EE, and integrity all experiencing substantial decreases, clearly affected by the destructive action of nebulization. After adding 0.1-10 mg/ml of HS15, all values before and after nebulization do not change significantly, especially the integrity of mRNA which is almost unaffected, thereby ensuring that the delivered mRNA can better exert its therapeutic effect in vivo. In the absence of HS15, significant increases in particle size, substantial declines in PDI, EE, and structural integrity were observed after nebulization, indicating severe damage from the nebulization process. However, with HS15 added at 0.1-10 mg/mL, no notable changes occurred in these parameters before and after nebulization. Especially, mRNA integrity remained almost unaffected, ensuring effective therapeutic delivery in vivo.

### 3) Stability Study

Samples prepared in Table 11 were stored at 4 °C, and particle size was measured after 7 days of storage to study stability.

TABLE 12
Stability information of samples

| | Particle size | |
|---|---|---|
| Sample name | (d · nm) | PDI |
| HS15-0.1-pre-7 d | 98.21 | 0.1233 |
| HS15-2-pre-7 d | 98.33 | 0.1793 |
| HS15-5-pre-7 d | 98.2 | 0.1701 |
| HS15-10-pre-7 d | 98.37 | 0.1662 |
| HS15-0-pre-7 d | 101.7 | 0.1602 |

The results show that after storage for a certain period, the particle size of the samples showed no significant changes, indicating that the introduction of HS15 does not compromise the storage stability of the LNPs.

### 4) Nebulization Time Study

We further investigated the impact of HS15 addition on nebulization time and process parameters. Here, “2% ethanol+10% propylene glycol” was abbreviated as “2+10” in the tables, corresponding to the formulation in Table 10 (2% ethanol+10% propylene glycol).

TABLE 13
Nebulization phenomenon

| Sample | Nebulization | | Nebulization |
|---|---|---|---|
| name | volume (μL) | Nebulization time | phenomenon |
| HS15-10-pre | 300 | 1 min 20 second | Smooth nebulization process |
| HS15-5-pre | 300 | 1 min 14 second | Smooth nebulization process |
| HS15-2-pre | 300 | 56 second | Smooth nebulization process |
| 2 + 10-pre | 300 | 2 min 45 second | Smooth nebulization process |
| pre | 300 | 56 second | Smooth nebulization process |

Conclusion: During nebulization, the addition of HS15 showed negligible impact on nebulization time, and the overall process proceeded smoothly. Compared to other systems containing surface tension-modulating agents (e.g., 2% ethanol+10% propylene glycol, abbreviated as 2+10 in Table 10), nebulization time was reduced by nearly 3 folds. Excessive nebulization time can adversely affect formulation stability and lead to poor patient compliance when developed into inhalable formulations. The inclusion of HS15 overcomes these drawbacks, demonstrating excellent technical effects.

By introducing molecules capable of reducing the surface tension of water into the LNP dispersion system, it was unexpectedly discovered that LNP prepared using this formulation exhibited resistance to shear forces during nebulization, maintained particle size and PDI unchanged before and after nebulization. Additionally, no nucleic acid leakage from the LNPs has occurred, ensuring encapsulation efficiency (EE) consistency. LNPs achieved high pulmonary deposition and gene transfection efficacy both before and after nebulization has overcome the technical barriers of commercial nebulized LNPs (poor shear resistance and instability). This innovation achieved unprecedented technical effects.

The foregoing descriptions and embodiments illustrate the basic principles, key features, and advantages of the present invention. Those skilled in the art should understand that the described examples are not intended to limit the invention in any form. Any technically equivalent substitutions or modifications derived from the principles of this invention shall fall within the scope of its protection.

1. A general formulation of nebulized inhaled lipid nanoparticles, wherein comprises a surface tension-lowering regulator added into a lipid nanoparticle dispersion system to increase the nebulization shear resisting ability of lipid nanoparticles.

2. The general formulation of nebulized inhaled lipid nanoparticles of claim 1, wherein comprises: a lipid nanoparticle dispersion system and a surface tension-lowering regulator added to the lipid nanoparticle dispersion system; the particle size range of the lipid nanoparticles is 70~160 nm; the mass-volume percentage range of the surface tension-lowering regulator in the lipid nanoparticle dispersion system is 0.01%~30%.

3. The general formulation of nebulized inhaled lipid nanoparticles of claim 2, wherein the lipid nanoparticle dispersion system comprises: lipid nanoparticles, and a system solution; the mass-volume percentage range of the lipid nanoparticles in the dispersion system is 0.0025%~10%, and the concentration range of the system solution is 0~1000 mM.

4. The general formulation of nebulized inhaled lipid nanoparticles of claim 3, wherein the lipid nanoparticle dispersion system comprises: lipid nanoparticles, a system solution, and a cryoprotectant; the mass-volume percentage range of the lipid nanoparticles in the dispersion system is 0.0025%~10%, the concentration range of the system solution is 0~1000 mM, and the mass-volume percentage range of the cryoprotectant is 0%~20%.

5. The general formulation of nebulized inhaled lipid nanoparticles of claim 3,
wherein the lipid nanoparticles comprise: ionizable lipid:helper lipid:structural lipid:polymer-conjugated lipid=30~60:5~40:20~40:0.1~15, by molar ratio.

6. The general formulation of nebulized inhaled lipid nanoparticles of claim 3, wherein the system solution comprises: normal saline, 4-(2-hydroxyethyl) piperazine-1-ethanesulfonic acid buffer (HEPES), tris(hydroxymethyl)aminomethane buffer (Tris), Tris-EDTA buffer, phosphate buffer (PB) and phosphate-buffered saline (PBS) buffer, Dulbecco's phosphate-buffered saline (DPBS), citrate buffer, sulfate buffer, carbonate buffer, acetate buffer, Tris-buffered saline with Tween (TBST), EDTA containing buffer and its sodium salt, and the combinations of one or more of the above.

7. The general formulation of nebulized inhaled lipid nanoparticles of claim 1, wherein the surface tension-lowering regulator comprises: ethanol, propylene glycol, benzyl alcohol, poloxamer 188, Tween-80, glycerol, polyoxyl-15 hydroxystearate, or the combinations of one or more of the above.

8. The general formulation of nebulized inhaled lipid nanoparticles of claim 7, wherein the surface tension-lowering regulator is ethanol, and the mass-volume percentage range of ethanol in the lipid nanoparticle dispersion system is 0.05~30%.

9. The general formulation of nebulized inhaled lipid nanoparticles of claim 7, wherein the surface tension-lowering regulator is propylene glycol, and the mass-volume percentage range of propylene glycol in the lipid nanoparticle dispersion system is 0.05~30%.

10. The general formulation of nebulized inhaled lipid nanoparticles of claim 7, wherein the surface tension-lowering regulator is a combination of propylene glycol and ethanol, the mass-volume percentage range of propylene glycol in the lipid nanoparticle dispersion system is 0.05~30%, and the mass-volume percentage range of ethanol in the lipid nanoparticle dispersion system is 0.05~30%.

11. The general formulation of nebulized inhaled lipid nanoparticles of claim 7, wherein the surface tension-lowering regulator is poloxamer 188, and the concentration range of poloxamer 188 in the lipid nanoparticle dispersion system is 0.5~10 mg/mL.

12. The general formulation of nebulized inhaled lipid nanoparticles of claim 7, wherein the surface tension-lowering regulator is a mixture of poloxamer 188 and ethanol, the concentration range of poloxamer 188 in the lipid nanoparticle dispersion system is 0.5~10 mg/mL, and the mass-volume percentage range of ethanol in the lipid nanoparticle dispersion system is 0.05~30%.

13. The general formulation of nebulized inhaled lipid nanoparticles of claim 7, wherein the surface tension-lowering regulator is Tween-80, and the mass-volume percentage range of Tween-80 in the lipid nanoparticle dispersion system is 0.01~2%.

14. The general formulation of nebulized inhaled lipid nanoparticles of claim 7, wherein the surface tension-lowering regulator is a mixture of Tween-80 and ethanol, the mass-volume percentage range of Tween-80 in the lipid nanoparticle dispersion system is 0.01~2%, and the mass-volume percentage range of ethanol in the lipid nanoparticle dispersion system is 0.05~30%.

15. The general formulation of nebulized inhaled lipid nanoparticles of claim 7, wherein the surface tension-lowering regulator is polyoxyl-15 hydroxystearate, and the concentration range of polyoxyl-15 hydroxystearate in the lipid nanoparticle dispersion system is 0.05-20 mg/ml, preferably 0.1-10 mg/ml, and more preferably 3-7.5 mg/ml.
